(19) United States
(12) Patent Application Publication
(10) Pub. No.: US 2006/0134636 A1
(43) Pub. Date: Jun. 22, 2006

(54) Standardization of growth conditions for human embryonic stem cells intended for use in regenerative medicine

(76) Inventors: Lawrence W. Stanton, Singapore (SG); Ralph Brandenberger, Menlo Park, CA (US); Elisa Brunette, Redwood City, CA (US); Joseph D. Gold, San Francisco, CA (US); John M. Irving, San Mateo, CA (US); Ramkumar Mandalam, Union City, CA (US); Michael Mok, Palo Alto, CA (US); Sandra E. Powell, Castro Valley, CA (US)

Correspondence Address: GERON CORPORATION, 230 CONSTITUTION DRIVE, MENLO PARK, CA, 94025, US

(21) Appl. No.: 10/804,822

(22) Filed: Mar. 19, 2004

Related U.S. Application Data
(63) Continuation-in-part of application No. 10/388,578, filed on Mar. 13, 2003.
(63) Continuation-in-part of application No. 10/389,431, filed on Mar. 13, 2003.
(63) Continuation-in-part of application No. PCT/US04/08883, filed on Mar. 13, 2004.
(63) Continuation-in-part of application No. PCT/EP04/02808, filed on Mar. 15, 2004.

(51) Int. Cl. C12Q 1/68; G01N 33/567; C12N 5/08; G01N 33/53

(52) U.S. Cl. 435/6; 435/366; 435/7.2

(57) ABSTRACT

This disclosure provides a system for qualifying embryonic stem cells intended for human therapy. A comprehensive sequencing project has identified important markers that are characteristic of undifferentiated pluripotent cells. Combinations of these markers have been used to screen feeder cells, media additives, and culture conditions that promote rapid expansion of stem cells without differentiation. By measuring undifferentiated stem cell markers, and markers formed by early progenitors such as stromal cells, the user can quantitate the proportion and extent of differentiation. This establishes standardized criteria for master cell banks and cell cultures that can then be used to produce therapeutic cell populations and medicaments for use in regenerative medicine.

### REFERENCE TO RELATED APPLICATIONS

This application is a continuation-in-part of pending U.S. utility application Ser. No. 10/388,578 (135/001) and Ser. No. 10/389,431 (135/002), both filed Mar. 13, 2003; PCT application designating the U.S. entitled “Genes That Are Up- or Down-Regulated During Differentiation of Human Embryonic Stem Cells” (135/200pct), filed Mar. 15, 2004; and PCT application designating the U.S. entitled “A Marker System for Characterizing Undifferentiated Human Embryonic Stem Cells” (135/300pct), filed Mar. 15, 2004.

The priority applications are hereby incorporated herein by reference with respect to markers that are up- or down-regulated during hES cell differentiation, and their use for developing and monitoring culture systems for hES cells and their equivalents.

### BACKGROUND

A promising development in the field of regenerative medicine has been the isolation and propagation of human stem cells from the early embryo. These cells have two very special properties: First, unlike other normal mammalian cell types, they can be propagated in culture almost indefinitely, providing a virtually unlimited supply. Second, they can be used to generate a variety of tissue types of interest as a source of replacement cells and tissues for use in therapy.

Thomson et al. (Science 282:114, 1998; U.S. Pat. No. 6,200,806) were the first to successfully isolate and propagate embryonic stem cells from human blastocysts. Gearhart and coworkers derived human embryonic germ cell lines from fetal gonadal tissue (Shamblott et al., Proc. Natl. Acad. Sci. USA 95:13726, 1998; U.S. Pat. No. 6,090,622).

International Patent Publication WO 99/20741 (Geron Corp.) describes methods and materials for the growth of primate-derived primordial stem cells. International Patent Publication WO 01/51616 (Geron Corp.) provides techniques for growth and differentiation of human pluripotent stem cells. An article by Xu et al. (Nature Biotechnology 19:971, 2001) describes feeder-free growth of undifferentiated human embryonic stem cells. Lebkowski et al. (Cancer J. 7 Suppl. 2:S83, 2001) discuss the culture, differentiation, and genetic modification of human embryonic stem cell for regenerative medicine applications. These publications report exemplary culture methods for propagating human embryonic stem cells in an undifferentiated state, and their use in preparing cells for human therapy.

Markers for identifying undifferentiated pluripotent stem cells include SSEA-4, Tra-1-60, and Tra-1-81 (Thomson et al. and Gearhart et al., supra). They also express human telomerase reverse transcriptase, and the POU transcription factor Oct 3/4 (WO 01/51616; Amit et al., Dev. Biol. 227:271, 2000; Xu et al., supra).

The following disclosure provides new markers and marker combinations that are effective means to establish and expand populations of undifferentiated pluripotent cells for commercial purposes.

### SUMMARY OF THE INVENTION

This invention ensues from a comprehensive sequencing project in which Geron Corporation identified a number of genes that are up- or down-regulated during the course of differentiation of early-stage pluripotent stem cells.

This patent disclosure provides a system of markers that is correlated with the presence or proportion of undifferentiated or differentiated cells in a population of cells, particularly human embryonic stem (hES) cells. The qualification procedure involves detecting or measuring markers preferentially expressed in undifferentiated hES cells, in combination with markers expressed preferentially after differentiation of the hES cells. For purposes of maintaining a relatively homogeneous, highly replicative cell population suitable for differentiation into any therapeutic cell type, the user will aim for cell populations and culture conditions that cause high level expression of the undifferentiated cell markers in most cells in the population, and low level or infrequent expression of the differentiated cell markers.

Markers that are preferentially expressed in undifferentiated hES cells are exemplified by Cripto, gastrin-releasing peptide (GRP) receptor, podocalyxin-like protein (PODXL), and traditional markers such as human telomerase reverse transcriptase (hTERT), Oct 3/4, SSEA-4, Tra-1-60 and Tra-1-81.

Markers preferentially expressed in differentiated cells can be found in early stage derivatives (such as stromal cells, embryoid body cells, or mixed progenitor cell populations), or more mature cell types. Exemplary differentiated cell markers include CD44, CD105 (endoglin), CD106 (VCAM-1), CD90 (Thy-1), STRO-1, Vimentin, and Human Thymus Stroma.

The markers can be detected or measured at the mRNA level (for example, by PCR amplification); or at the antigen expression level (for example, by flow cytometry or immunocytochemistry). Assessing the quality of the cell population as a whole can be done by determining positive expression of the undifferentiated cell markers, and lack of expression of the stromal cell markers.

The marker system of this invention can be used to assess the ability of a soluble factor or culture medium to maintain hES cells in an undifferentiated state from said marker expression; or to determine the suitability of an undifferentiated hES cell population for preparing differentiated cells for human administration.

Aspects of the invention include both the process for determining and interpreting the markers expressed by the cell population, and the reagent system useful for performing tests of this kind for any desirable purpose. Other aspects of the invention will be apparent from the description that follows.

By employing the marker system provided in this disclosure, the user can create a master cell bank or cell production facility that allows the manufacture of cell populations and medicaments with standardized criteria suitable for human therapy, or use in drug screening.

### DRAWINGS

FIG. 1 shows the profile of genes preferentially expressed in undifferentiated pluripotent stem cells, upon preliminary differentiation of the cells by culturing in retinoic acid or DMSO. Level of gene expression at the mRNA level was measured by real-time PCR assay. Any of the genes showing substantial down-regulation upon differentiation can be used to characterize the undifferentiated cell population, and culture methods suitable for maintaining them in an undifferentiated state.

FIG. 2 shows the level of expression of five genes in hES cells, compared with fully differentiated cells. This five-marker panel provides robust qualification of the undifferentiated phenotype.

FIG. 3 show results of an experiment in which hES cells of the H1 line were maintained for multiple passages in different media. Medium conditioned with feeder cells provides factors effective to allow hES cells to proliferate in culture without differentiating. However, culturing in unconditioned medium leads to decreased percentage of cells expressing CD9, and the classic hES cell marker SSEA-4.

FIG. 4 illustrates the sensitivity of hTERT, Oct 3/4, Cripto, GRP receptor, and podocalyxin-like protein (measured by real-time PCR) as a means of determining the degree of differentiation of the cells. After multiple passages in unconditioned medium, all five markers show expression that has been downregulated by 10 to 10⁴-fold.

FIG. 5 shows results of an experiment in which the hES cell line H1 was grown on different feeder cell lines: mEF=mouse embryonic fibroblasts; hMSC=human mesenchymal stem cells; UtSMC=uterine smooth muscle cells; WI-38=human lung fibroblasts. As monitored using Cripto, the hMSC is suitable for use as feeder cells to promote hES cell proliferation without differentiation.

FIG. 6 shows results of an experiment in which different media were tested for their ability to promote growth of hES cells without proliferation. The test media were not preconditioned, but supplemented with 8-40 ng/mL bFGF, with or without stem cell factor, Flt3 ligand, or LIF. Effective combinations of factors (Conditions 4 to 8) were identified by following the undifferentiated phenotype using the markers of this invention. Alterations in expression profiles were temporary and reversible, showing that the cells are still undifferentiated.

FIG. 7 shows analysis of the undifferentiated hES cell markers SSEA-4, TRA 1-60 and Oct-4 by antibody staining and flow cytometry. Oct-4 is detected by permeabilizing the cells before staining.

FIG. 8 shows the results of the immunocytochemical analysis for stromal cell markers CD44, STRO-1 and Vimentin, which label cells in the hES cell culture that have undergone differentiation.

FIG. 9 shows the relative gene expression levels for cell populations in which undifferentiated hES cells were mixed with BJ fibroblasts in increasing amounts.

### DETAILED DESCRIPTION

The propensity of pluripotent stem cells to differentiate spontaneously has made it challenging for investigators to work with these cells. Consistent cultures of undifferentiated stem cells are required to compare results obtained from multiple experiments performed within or between laboratories. Unfortunately, morphological characterization is subjective and especially difficult for cultures that often contain 10-20% differentiated cells. Nevertheless, having a set of standardized criteria will be important in qualifying these cells for use in clinical therapy.

The marker system identified in this disclosure provides the basis for establishing these standards. 148,453 different transcripts were amplified and sequenced from undifferentiated human embryonic stem cells, and three types of progeny. As a result of this sequencing effort, 532 genes were identified having substantially higher EST counts in undifferentiated cells, and 142 genes were identified having substantially higher EST counts after differentiation. Other differentially expressed genes were identified by microarray analysis of undifferentiated cells, compared with cells at the beginning of the differentiation process.

The system provided by this invention can be used to qualify populations of pPS cells, both in terms of the phenotype of the undifferentiated cells, and the proportion and phenotype of contaminating differentiated cells that may be present. Culture systems have been identified and protocols have been developed to expand cultures of undifferentiated cells and produce commercially viable quantities of cells for use in research, drug screening, and regenerative medicine.

### DEFINITIONS

“Pluripotent Stem cells” (pPS cells) are pluripotent cells that have the characteristic of being capable under appropriate conditions of producing progeny of several different cell types that are derivatives of all of the three germinal layers (endoderm, mesoderm, and ectoderm), according to a standard art-accepted test, such as the ability to form a teratoma in 8-12 week old SCID mice. The term includes both established lines of stem cells of various kinds, and cells obtained from primary tissue that are pluripotent in the manner described. For the purposes of this disclosure, the pPS cells are not embryonal carcinoma (EC) cells, and are not derived from a malignant source. It is desirable (but not always necessary) that the cells be euploid. Exemplary pPS cells are obtained from embryonic or fetal tissue at any time after fertilization.

“Human Embryonic Stem cells” (hES cells) are pluripotent stem cells derived from a human embryo in the blastocyst stage, or human pluripotent cells produced by artificial means (such as by nuclear transfer) that have equivalent characteristics. Exemplary derivation procedures and features are provided in a later section.

hES cell cultures are described as “undifferentiated” when a substantial proportion (at least 20%, and possibly over 50% or 80%) of stem cells and their derivatives in the population display morphological characteristics of undifferentiated cells, distinguishing them from differentiated cells of embryo or adult origin. It is understood that colonies of undifferentiated cells within the population will often be surrounded by neighboring cells that are differentiated. It is also understood that the proportion of cells displaying the undifferentiated phenotype will fluctuate as the cells proliferate and are passaged from one culture to another. Cells are recognized as proliferating in an undifferentiated state when they go through at least 4 passages and/or 8 population doublings while retaining at least about 50%, or the same proportion of cells bearing characteristic markers or morphological characteristics of undifferentiated cells.

A “differentiated cell” is a cell that has progressed down a developmental pathway, and includes lineage-committed progenitor cells and terminally differentiated cells.

“Feeder cells” or “feeders” are terms used to describe cells of one type that are co-cultured with cells of another type, to provide an environment in which the cells of the second type can grow. hES cell populations are said to be “essentially free” of feeder cells if the cells have been grown through at least one round after splitting in which fresh feeder cells are not added to support the growth of pPS cells.

The term “embryoid bodies” refers to aggregates of differentiated and undifferentiated cells that appear when pPS cells overgrow in monolayer cultures, or are maintained in suspension cultures. Embryoid bodies are a mixture of different cell types, typically from several germ layers, distinguishable by morphological criteria and cell markers detectable by immunocytochemistry.

The term “stromal cell” as it is used in this disclosure means a cell differentiated within a culture of pPS cells that is no longer pluripotent, and may have a fibrous nature reminiscent of connective tissue cells. Stromal cells can be detected in pPS cultures by expression of least one, and usually two or more markers selected from CD44, CD105, VCAM-1, Thy-1, STRO-1, Vimentin, and Human Thymus Stroma.

A cell “marker” is any phenotypic feature of a cell that can be used to characterize it or discriminate it from other cell types. A marker of this invention may be a protein (including secreted, cell surface, or internal proteins; either synthesized or taken up by the cell); a nucleic acid (such as an mRNA, or enzymatically active nucleic acid molecule) or a polysaccharide. Included are determinants of any such cell components that are detectable by antibody, lectin, probe or nucleic acid amplification reaction that are specific for the cell type of interest. The markers can also be identified by a biochemical or enzyme assay that depends on the function of the gene product. Associated with each marker is the gene that encodes the transcript, and the events that lead to marker expression.

A marker is said to be “preferentially expressed” in an undifferentiated or differentiated cell population, if it is expressed at a level that is at least 10 times higher (in terms of total gene product measured in an antibody or PCR assay) or 10 times more frequently (in terms of positive cells in the population). Markers that are expressed 100, 1,000, or 10,000 times higher or more frequently are increasingly more preferred.

The terms “polynucleotide” and “nucleic acid” refer to a polymeric form of nucleotides of any length. Included are genes and gene fragments, mRNA, cDNA, plasmids, viral and non-viral vectors and particles, nucleic acid probes, amplification primers, and their chemical equivalents. As used in this disclosure, the term polynucleotide refers interchangeably to double- and single-stranded molecules. Unless otherwise specified, any embodiment of the invention that is a polynucleotide encompasses both a double-stranded form, and each of the two complementary single-stranded forms known or predicted to make up the double-stranded form.

A cell is said to be “genetically altered” or “transfected” when a polynucleotide has been transferred into the cell by any suitable means of artificial manipulation, or where the cell is a progeny of the originally altered cell that has inherited the polynucleotide.

A “control element” or “control sequence” is a nucleotide sequence involved in an interaction of molecules that contributes to the functional regulation of a polynucleotide, including replication, duplication, transcription, splicing, translation, or degradation of the polynucleotide. “Operatively linked” refers to an operative relationship between genetic elements, in which the function of one element influences the function of another element. For example, an expressible encoding sequence may be operatively linked to a promoter that drives gene transcription.

The term “antibody” as used in this disclosure refers to both polyclonal and monoclonal antibody. The ambit of the term deliberately encompasses not only intact immunoglobulin molecules, but also such fragments and derivatives of immunoglobulin molecules that retain a desired binding specificity.

General Techniques

Methods in molecular genetics and genetic engineering are described generally in the current editions of Molecular Cloning: A Laboratory Manual, (Sambrook et al.); Oligonucleotide Synthesis (M. J. Gait, ed.); Animal Cell Culture (R. I. Freshney, ed.); Gene Transfer Vectors for Mammalian Cells (Miller & Calos, eds.); Current Protocols in Molecular Biology and Short Protocols in Molecular Biology, 3^{rd} Edition (F. M. Ausubel et al., eds.); and Recombinant DNA Methodology (R. Wu ed., Academic Press). Antibody production is described in Basic Methods in Antibody Production and Characterization (Howard & Bethell eds., CRC Press, 2000).

A survey of relevant techniques is provided in such standard texts as DNA Sequencing (A. E. Barron , John Wiley, 2002), and DNA Microarrays and Gene Expression (P. Baldi et al., Cambridge U. Press, 2002). For a description of the molecular biology of cancer, the reader is referred to Principles of Molecular Oncology (M. H. Bronchud et al. eds., Humana Press, 2000); The Biological Basis of Cancer (R. G. McKinnel et al. eds., Cambridge University Press, 1998); and Molecular Genetics of Cancer (J. K. Cowell ed., Bios Scientific Publishers, 1999).

Sources of Stem Cells

This invention is based on observations made with established lines of hES cells. The markers are suitable for identifying, characterizing, and manipulating related types of undifferentiated pluripotent cells. They are also suitable for use with pluripotent cells obtained from primary embryonic tissue, without first establishing an undifferentiated cell line. It is contemplated that the markers described in this application will in general be useful for other types of pluripotent cells, including embryonic germ cells (U.S. Pat. Nos. 6,090,622 and 6,251,671), and ES and EG cells from other mammalian species, such as non-human primates.

Embryonic Stem Cells

Embryonic stem cells can be isolated from blastocysts of members of primate species (U.S. Pat. No. 5,843,780; Thomson et al., Proc. Natl. Acad. Sci. USA 92:7844, 1995). Human embryonic stem (hES) cells can be prepared from human blastocyst cells using the techniques described by Thomson et al. (U.S. Pat. No. 6,200,806; Science 282:1145, 1998; Curr. Top. Dev. Biol. 38:133 ff., 1998) and Reubinoff et al, Nature Biotech. 18:399, 2000. Equivalent cell types to hES cells include their pluripotent derivatives, such as primitive ectoderm-like (EPL) cells, outlined in WO 01/51610 (Bresagen).

hES cells can be obtained from human preimplantation embryos (Thomson et al., Science 282:1145, 1998). Alternatively, in vitro fertilized (IVF) embryos can be used, or one-cell human embryos can be expanded to the blastocyst stage (Bongso et al., Hum Reprod 4: 706, 1989). Embryos are cultured to the blastocyst stage, the zona pellucida is removed, and the inner cell masses are isolated (for example, by immunosurgery using rabbit anti-human spleen cell antiserum). The intact inner cell mass is plated on mEF feeder layers, and after 9 to 15 days, inner cell mass derived outgrowths are dissociated into clumps. Growing colonies having undifferentiated morphology are dissociated into clumps, and replated. ES-like morphology is characterized as compact colonies with apparently high nucleus to cytoplasm ratio and prominent nucleoli. Resulting ES cells are then routinely split every 1-2 weeks. Clump sizes of about 50 to 100 cells are optimal.

Propagation of pPS Cells in an Undifferentiated State

pPS cells can be propagated continuously in culture, using culture conditions that promote proliferation while inhibiting differentiation. Exemplary serum-containing ES medium is made with 80% DMEM (such as Knock-Out DMEM, Gibco), 20% of either defined fetal bovine serum (FBS, Hyclone) or serum replacement (US 20020076747 A1, Life Technologies Inc.), 1% non-essential amino acids, 1 mM L-glutamine, and 0.1 mM β-mercaptoethanol.

Traditionally, ES cells are cultured on a layer of feeder cells, typically fibroblasts derived from embryonic or fetal tissue (Thomson et al., Science 282:1145, 1998). Scientists at Geron have discovered that pPS cells can be maintained in an undifferentiated state even without feeder cells. The environment for feeder-free cultures includes a suitable culture substrate, particularly an extracellular matrix such as Matrigel® or laminin. The pPS cells are plated at >15,000 cells cm⁻² (optimally 90,000 cm⁻² to 170,000 cm⁻²). Typically, enzymatic digestion is halted before cells become completely dispersed (say, ˜5 min with collagenase IV). Clumps of ˜10 to 2,000 cells are then plated directly onto the substrate without further dispersal. Alternatively, the cells can be harvested without enzymes before the plate reaches confluence by incubating ˜5 min in a solution of 0.5 mM EDTA in PBS. After washing from the culture vessel, the cells are plated into a new culture without further dispersal. In a further illustration, confluent hES cells cultured in the absence of feeders are removed from the plates by incubating with a solution of 0.05% (wt/vol) trypsin (Gibco) and 0.053 mM EDTA for 5-15 min at 37° C. The remaining cells in the plate are removed and the cells are triturated into a suspension comprising single cells and small clusters, and then plated at densities of 50,000-200,000 cells cm⁻² to promote survival and limit differentiation.

Feeder-free cultures are supported by a nutrient medium containing factors that promote proliferation of the cells without differentiation (WO 99/20741). Such factors may be introduced into the medium by culturing the medium with cells secreting such factors, such as irradiated (˜4,000 rad) primary mouse embryonic fibroblasts, telomerized mouse fibroblasts, or fibroblast-like cells derived from pPS cells (U.S. Pat. No. 6,642,048). Medium can be conditioned by plating the feeders in a serum free medium such as KO DMEM supplemented with 20% serum replacement and 4 ng/mL bFGF. Medium that has been conditioned for 1-2 days is supplemented with further bFGF, and used to support pPS cell culture for 1-2 days (WO 01/51616; Xu et al., Nat. Biotechnol. 19:971, 2001). Alternatively, fresh or non-conditioned medium can be used, which has been supplemented with added factors (like a fibroblast growth factor or forskolin) that promote proliferation of the cells in an undifferentiated form (WO 03/020920). Using the marker system of this invention to identify or optimize culture methods for hES cells is illustrated in Example 5.

Under the microscope, ES cells appear with high nuclear/cytoplasmic ratios, prominent nucleoli, and compact colony formation with poorly discernable cell junctions. Conventional markers for hES cells are stage-specific embryonic antigen (SSEA) 3 and 4, and markers detectable using antibodies Tra-1-60 and Tra-1-81 (Thomson et al., Science 282:1145, 1998). Differentiation of pPS cells in vitro results in the loss of SSEA-4, Tra-1-60, and Tra-1-81 expression, and increased expression of SSEA-1.

Markers for Undifferentiated DPS Cells and Their Differentiated Progeny

Under the microscope, ES cells appear with high nuclear/cytoplasmic ratios, prominent nucleoli, and compact colony formation with poorly discernable cell junctions. Conventional markers for hES cells are stage-specific embryonic antigen (SSEA) 3 and 4, and markers detectable using antibodies Tra-1-60 and Tra-1-81 (Thomson et al., Science 282:1145, 1998). Differentiation of pPS cells in vitro results in the loss of SSEA-4, Tra-1-60, and Tra-1-81 expression, and increased expression of SSEA-1.

The tables and description provided later in this disclosure provide new markers that distinguish undifferentiated pPS cells from their differentiated progeny.

Expression libraries were made from ES cells (WO 01/51616), embryoid bodies (WO 01/51616), and cells differentiated towards the hepatocyte (WO 01/81549) or neural cell (WO 01/88104) lineage. mRNA was reverse transcribed and amplified, producing expressed sequence tags (ESTs) occurring in frequency proportional to the level of expression in the cell type being analyzed. The ESTs were subjected to automatic sequencing, and counted according to the corresponding unique (non-redundant) transcript. A total of 148,453 non-redundant transcripts were represented in each of the 4 libraries. Genes were then identified as having a differential expression pattern if the number of EST counts of the transcript was statistically different between the libraries being compared.

In a parallel set of experiments, mRNA from each of the cell types was analyzed for binding to a broad-specificity EST-based microarray, performed according to the method described in WO 01/51616. Genes were identified as having a differential expression pattern if they showed a comparatively different signal on the microarray.

Significant expression differences determined by EST sequencing, microarray analysis, or other observations were confirmed by real-time PCR analysis. The mRNA was amplified by PCR using specific forward and reverse primers designed from the GenBank sequence, and the amplification product was detected using labeled sequence-specific probes. The number of amplification cycles required to reach a threshold amount was then compared between different libraries.

Now that genes have been identified that are up-regulated or down-regulated upon differentiation, a number of commercial applications of these markers will be apparent to the skilled reader. The sections that follow provide non-limiting illustrations of how some of these embodiments can be implemented.

Use of Cell Markers to Characterize pPS Cells and Their Differentiated Progeny

The markers provided in this disclosure can be used as a means to identify both undifferentiated and differentiated cells—either a population as a whole, or as individual cells within a population. This can be used to evaluate the expansion or maintenance of pre-existing cell populations, or to characterize the pluripotent nature (or lineage commitment) of newly obtained populations.

Expression of single markers in a test cell will provide evidence of undifferentiated or differentiated phenotype, according to the expression pattern listed later in this disclosure. A plurality of markers (such as any 2, 3, 4, 5, 6, 8, 10, 12, 15, or 20 markers selected from amongst Tables 2-5 and 7) will provide a more detailed assessment of the characteristics of the cell. Expression of genes that are down-regulated and/or lack of expression of genes that are up-regulated upon differentiation correlates with a differentiated phenotype. Expression of genes that are up-regulated and/or lack of expression of genes that are down-regulated upon differentiation correlates with an undifferentiated phenotype. The markers newly identified in this disclosure may be analyzed together (with or without markers that were previously known) in any combination effective for characterizing the cell status or phenotype.

Exemplary combinations of undifferentiated markers are provided elsewhere in this disclosure (e.g., Tables 2 and 4). For determining the undifferentiated cell phenotype, combinations of markers like Cripto, gastrin-releasing peptide (GRP) receptor, podocalyxin-like protein (PODXL), and human telomerase reverse transcriptase (hTERT) are effective, either alone, or in combination with cell surface markers like SSEA-3, SSEA-4, Tra-1-60 and Tra-1-81, or intracellular markers like Oct 3/4. For determining differentiated cells, any marker can be used that is characteristic of contaminating cells that may be present (e.g., Tables 3 and 5). Depending on culture conditions, early stage non-specific hES cell differentiation generates cells having characteristics of stromal cells, fibroblasts, mesenchymal cells, embryoid body cells, and other cell types. Markers of stromal cells of current interest include Vimentin, CD44, and other markers listed in Table 7. A combination of markers characteristic of several types of cells can also be used, as long as they are preferentially expressed in differentiated cells.

The skilled reader may want to monitor the presence of other cell types of interest, using markers for particular progenitor or terminally differentiated cells. Tissue-specific markers are listed in WO 01/81549 (hepatocytes), WO 01/88104 (neural cells), PCT/US02/20998 (osteoblasts and mesenchymal cells), PCT/US02/22245 (cardiomyocytes), PCT/US02/39091 (hematopoietic cells), PCT/US02/39089 (islet cells), and PCT/US02/39090 (chondrocytes).

Tissue-specific markers can be detected using any suitable immunological technique—such as flow cytochemistry for cell-surface markers, or immunocytochemistry (for example, of fixed cells or tissue sections) for intracellular or cell-surface markers. Expression of a cell-surface antigen is defined as positive if a significantly detectable amount of antibody will bind to the antigen in a standard immunocytochemistry or flow cytometry assay, optionally after fixation of the cells, and optionally using a labeled secondary antibody or other conjugate to amplify labeling.

The expression of tissue-specific gene products can also be detected at the mRNA level by Northern blot analysis, dot-blot hybridization analysis, or by reverse transcriptase initiated polymerase chain reaction (RT-PCR) using sequence-specific primers in standard amplification methods. See U.S. Pat. No. 5,843,780 for further details. Sequence data for particular markers listed in this disclosure can be obtained from public databases such as GenBank.

These and other suitable assay systems are described in standard reference texts, such as the following: PCR Cloning Protocols, 2^{nd} Ed (James & Chen eds., Humana Press, 2002); Rapid Cycle Real-Time PCR: Methods and Applications (C. Wittwer et al. eds., Springer-Verlag NY, 2002); Immunoassays: A Practical Approach (James Gosling ed., Oxford Univ Press, 2000); Cytometric Analysis of Cell Phenotype and Function (McCarthy et al. eds., Cambridge Univ Press, 2001). Reagents for conducting these assays, such as nucleotide probes or primers, or specific antibody, can be packaged in kit form, optionally with instructions for the use of the reagents in the characterization or monitoring of pPS cells, or their differentiated progeny.

Use of Cell Markers to Assess and Manipulate Culture Conditions

The markers and marker combinations of this invention provide a system for monitoring undifferentiated pPS cells and their differentiated progeny in culture. This system can be used as a quality control, to compare the characteristics of undifferentiated pPS cells between different passages or different batches. It can also be used to assess a change in culture conditions, to determine the effect of the change on the undifferentiated cell phenotype.

Where the object is to produce undifferentiated cells, a decrease in the level of expression of an undifferentiated marker because of the alteration by 3-, 10-, 25-, 100- and 1000-fold is progressively less preferred. Corresponding increases in marker expression may be more beneficial. Moderate decreases in marker expression may be quite acceptable within certain boundaries, if the cells retain their ability to form progeny of all three germ layers is retained, and/or the level of the undifferentiated marker is relatively restored when culture conditions are returned to normal.

In this manner, the markers of this invention can be used to evaluate different feeder cells, extracellular matrixes, base media, additives to the media, culture vessels, or other features of the culture environment. Once an optimized culture method has been validated, the marker system can then be used to monitor the quality of the cells produced on an ongoing basis.

### The Examples that Follow are Provided for Further Illustration, and are not Meant to Limit the Claimed Invention

### EXAMPLES

### Example 1

### An EST Database of Undifferentiated hES Cells and their Differentiated Progeny

cDNA libraries were prepared from human embryonic stem (hES) cells cultured in undifferentiated form. cDNA libraries were also prepared from progeny, subject to non-specific differentiation as embryoid bodies (EBs), or taken through the preliminary stages of established differentiation protocols for neurons (preNEU) or hepatocytes (preHEP).

The hES cell lines H1, H7, and H9 were maintained under feeder-free conditions. Cultures were passaged every 5-days by incubation in 1 mg/mL collagenase IV for 5-10 min at 37° C., dissociated and seeded in clumps at 2.5 to 10×10⁵ cells/well onto Matrigel™-coated six well plates in conditioned medium supplemented with 8 mg/mL bFGF. cDNA libraries were made after culturing for 5 days after the last passage.

EBs were prepared as follows. Confluent plates of undifferentiated hES cells were treated briefly with collagenase IV, and scraped to obtain small clusters of cells. Cell clusters were resuspended in 4 mL/well differentiation medium (KO DMEM containing 20% fetal bovine serum in place of 20% SR, and not preconditioned) on low adhesion 6-well plates (Costar). After 4 days in suspension, the contents of each well was transferred to individual wells pre-coated with gelatin. Each well was re-fed with 3 mL fresh differentiation medium every two days after replating. Cells were used for the preparation of cytoplasmic RNA on the eighth day after plating.

PreHEP cells were prepared based on the hepatocyte differentiation protocol described in WO 01/81549. Confluent wells of undifferentiated cells were prepared, and medium was changed to KO DMEM plus 20% SR+1% DMSO. The medium was changed every 24 h, and cells were used for preparation of cytoplasmic RNA on day 5 of DMSO treatment.

PreNEU cells were prepared based on the neural differentiation protocol described in WO 01/88104. hES cells of the H7 line (p29) were used to generate EBs as described above except that 10 μM all-trans RA was included in the differentiation medium. After 4 days in suspension, EBs were transferred to culture plate precoated with poly-L-lysine and laminin. After plating, the medium was changed to EPFI medium. Cells were used for the preparation of cytoplasmic RNA after 3 days of growth in EPFI.

Partial 5′ end sequences (an expressed sequence tag, or EST) were determined by conventional means for independent clones derived from each cDNA library. Overlapping ESTs were assembled into conjoined sequences.

TABLE 1
Non-redundant EST sequences

| | Number |
|---|---|
| Library | of ESTs |
| hESC | 37,081 |
| EB | 37,555 |
| preHEP | 35,611 |
| preNEU | 38,206 |
| Total | 148,453 |

All of the stem cell lines used for preparation of the expression libraries were originally isolated and initially propagated on mouse feeder cells. Accordingly, the libraries were analyzed to determine whether they were contaminated with murine retroviruses that had shed from the feeder cells and subsequently infected the stem cells. Three complete viral genomes were used in a BLAST search: Moloney murine leukemia virus, Friend murine leukemia virus, and murine type C retrovirus. No matches with a high score were found against any of the ESTs.

The sequences were then compared to the Unigene database of human genes. ESTs that were at least 98% identical, over a stretch of at least 150 nucleotides each, to a common reference sequence in Unigene, were assumed to be transcribed from the same gene, and placed into a common assembly. The complete set of 148,453 ESTs collapsed to a non-redundant set of 32,764 assemblies.

### Example 2

### Selection of Marker Genes Specific for Undifferentiated and Differentiated cells

Candidate markers were selected from a database based on the imputed level of gene expression. The frequency of ESTs for any particular gene correlates with the abundance of that mRNA in the cells used to generate the cDNA library. Thus, a comparison of frequencies of ESTs among the libraries indicates the relative abundance of the associated mRNA in the different cell types.

Candidate molecular markers were selected from the expressed gene (EST) database from their greater abundance in undifferentiated hES cells, relative to differentiated hES cells. Genes were identified as having a differential expression pattern (being up- or down-regulated) during the differentiation process, if the count of ESTs sequenced in the undifferentiated cells was substantially different from the sum of ESTs in the three differentiated libraries.

Oct 3/4 (a POU domain-containing transcription factor) and telomerase reverse transcriptase (hTERT) are known to be expressed preferentially in undifferentiated hES cells (WO 01/51616). Other genes suitable for characterizing or manipulating the undifferentiated phenotype are those that are down-regulated upon differentiation with a significance of p≦0.05, as determined by the Fisher Exact Test (explained below). 193 genes were found to have 4-fold more ESTs in hES cells, relative to each of the three cell types. 532 genes were found that were 2-fold greater hES cells, with a confidence of over 95% as determined by the Fisher Exact Test, relative to the sum of ESTs of the three cell types (minimum of 4 ESTs in hES cells). The following markers are of particular interest:

TABLE 2
EST Frequency of Genes that are Down-regulated upon Differentiation of hES cells

| | | | EST counts | | | |
|---|---|---|---|---|---|---|
| Geron ID | GenBank ID | Name | ES | EB | preHEP | preNEU |
| GA_10902 | NM_024504 | Pr domain containing 14 (PRDM14) | 12 | 1 | 0 | 0 |
| GA_11893 | NM_032805 | Hypothetical protein FLJ14549 | 25 | 0 | 0 | 0 |
| GA_12318 | NM_032447 | Fibrillin3 | 6 | 0 | 0 | 0 |
| GA_1322 | NM_000142 | Fibroblast growth factor receptor 3 precursor | 9 | 1 | 5 | 1 |
| | | (FGFR-3) | | | | |
| GA_34679 | NM_002015 | Forkhead box o1a (FOXO1a) | 4 | 0 | 1 | 1 |
| GA_1470 | NM_003740 | potassium channel, subfamily K, member 5 | 4 | 0 | 0 | 1 |
| | | (KCNK5), mRNA | | | | |
| GA_1674 | NM_002701 | Octamer-Binding Transcription Factor 3a | 24 | 1 | 2 | 0 |
| | | (OCT-3A) (OCT-4) | | | | |
| GA_2024 | NM_003212 | Teratocarcinoma-derived growth factor 1 | 20 | 1 | 0 | 0 |
| | | (CRIPTO) | | | | |
| GA_2149 | NM_003413 | Zic family member 3 (ZIC3) | 7 | 0 | 1 | 0 |
| GA_2334 | NM_000216 | Kallmann syndrome 1 sequence (KAL1) | 5 | 0 | 1 | 0 |
| GA_23552 | NM_152742 | hypothetical protein DKFZp547M109 | 6 | 0 | 1 | 2 |
| | | (DKFZp547M109), mRNA | | | | |
| GA_2356 | NM_002851 | Protein tyrosine phosphatase, receptor-type, | 10 | 0 | 0 | 0 |
| | | z polypeptide 1 (PTPRZ1), | | | | |
| GA_2357 | NM_001670 | Armadillo repeat protein deleted in | 6 | 0 | 0 | 0 |
| | | velo-cardio-facial syndrome (ARVCF) | | | | |
| GA_23578 | BM454360 | AGENCOURT_6402318 NIH_MGC_85 | 6 | 0 | 0 | 0 |
| | | Homo sapiens cDNA clone IMAGE: 5497491 | | | | |
| | | 5′, mRNA sequence | | | | |
| GA_2367 | NM_003923 | Forkhead box H1 (FOXH1) | 5 | 0 | 0 | 0 |
| GA_2436 | NM_004329 | Bone morphogenetic protein receptor, type Ia | 7 | 3 | 1 | 1 |
| | | (BMPR1A)(ALK-3) | | | | |
| GA_2442 | NM_004335 | Bone marrow stromal antigen 2 (BST-2) | 13 | 0 | 2 | 3 |
| GA_2945 | NM_005232 | Ephrin type-a receptor 1 (EPHA1) | 5 | 1 | 1 | 1 |
| GA_2962 | NM_005314 | Gastrin-releasing peptide receptor (GRP-R) | 4 | 0 | 0 | 0 |
| GA_2988 | NM_005397 | Podocalyxin-like (PODXL) | 59 | 23 | 5 | 8 |
| GA_3337 | NM_006159 | NELL2 (nel-like protein 2) | 5 | 3 | 2 | 0 |
| GA_3559 | NM_005629 | Solute carrier family 6, member 8 (SLC6A8) | 5 | 1 | 0 | 1 |
| GA_3898 | NM_006892 | DNA (cytosine-5-)-methyltransferase 3 beta | 49 | 2 | 3 | 1 |
| | | (DNMT3B) | | | | |
| GA_5391 | NM_002968 | Sal-like 1 (SALL1), | 7 | 1 | 1 | 0 |
| GA_33680 | NM_016089 | Krab-zinc finger protein SZF1-1 | 15 | 0 | 1 | 0 |
| GA_36977 | NM_020927 | KIAA1576 protein | 9 | 2 | 1 | 0 |
| GA_8723 | NM_152333 | Homo sapiens chromosome 14 open reading | 14 | 1 | 1 | 3 |
| | | frame 69 (C14orf69), mRNA | | | | |
| GA_9167 | AF308602 | Notch 1 (N1) | 6 | 2 | 1 | 0 |
| GA_9183 | NM_007129 | Homo sapiens Zic family member 2 (odd- | 8 | 1 | 1 | 0 |
| | | paired homolog, Drosophila) (ZIC2), mRNA | | | | |
| GA_35037 | NM_004426 | Homo sapiens polyhomeotic-like 1 | 34 | 9 | 5 | 4 |
| | | (Drosophila) (PHC1), mRNA | | | | |

Only one EST for hTERT was identified in undifferentiated hES cells and none were detected from the differentiated cells, which was not statistically significant. Thus, potentially useful markers that are expressed at low levels could have been omitted in this analysis, which required a minimum of four ESTs. It would be possible to identify such genes by using other techniques described elsewhere in this disclosure.

Three genes were observed from EST frequency queries that were of particular interest as potentially useful markers of hES cells. They were Teratocarcinoma-derived growth factor (Cripto), Podocalyxin-like (PODXL), and gastrin-releasing peptide receptor (GRPR). These genes were not only more abundant in undifferentiated cells, relative to differentiated hES cells, but also encoded for proteins expressed on the surface of cells. Surface markers have the added advantage that they could be easily detected with immunological reagents. ESTs for Cripto and GRPR were quite restricted to hES cells, with one or zero ESTs, respectively, scored in any of the differentiated cells. PODXL ESTs were detected in all 4-cell types, but substantially fewer (2.5×-12×) in differentiated cells. All three markers retained a detectable level of expression in differentiated cultures of hES cells. There may be a low level of expression of these markers in differentiated cells, or the expression detected may be due to a small proportion of undifferentiated cells in the population. GABA(A) receptor, Lefty B, Osteopontin, Thy-1 co-transcribed, and Solute carrier 21 are other significant markers of the undifferentiated phenotype.

By similar reasoning, genes that show a higher frequency of ESTs in differentiated cells can be used as specific markers for differentiation. ESTs that are 2-fold more abundant in the sum of all three differentiated cell types (EBs, preHEP and preNEU cells) and with a p-value≦0.05 as determined by the Fisher Exact Test, compared with undifferentiated hES cells are candidate markers for differentiation down multiple pathways. ESTs that are relatively abundant in only one of the differentiated cell types are candidate markers for tissue-specific differentiation. The following markers are of particular interest:

TABLE 3
EST Frequency of Genes that are Upregulated upon Differentiation

| | | | EST counts | | | |
|---|---|---|---|---|---|---|
| Geron ID | GenBank ID | Name | ES | EB | preHEP | preNEU |
| GA_35463 | NM_024298 | Homo sapiens leukocyte receptor cluster (LRC) | 0 | 4 | 9 | 8 |
| | | member 4 (LENG4), mRNA | | | | |
| GA_10492 | NM_006903 | Inorganic pyrophosphatase (PPASE) | 0 | 5 | 5 | 6 |
| GA_38563 | NM_021005 | Homo sapiens nuclear receptor subfamily 2, | 0 | 9 | 8 | 9 |
| | | group F, member 2 (NR2F2), mRNA | | | | |
| GA_38570 | NM_001844 | Collagen, type II, alpha 1 (COL2A1), transcript | | 15 | 31 | 5 |
| | | variant 1 | | | | |
| GA_1476 | NM_002276 | Keratin type I cytoskeletal 19 (cytokeratin 19) | 1 | 26 | 14 | 38 |
| GA_34776 | NM_002273 | Keratin type II cytoskeletal 8 (cytokeratin 8) | 9 | 71 | 144 | 156 |
| | | (CK 8) | | | | |
| GA_1735 | NM_002806 | Homo sapiens proteasome (prosome, | 1 | 7 | 7 | 8 |
| | | macropain) 26S subunit, ATPase, 6 (PSMC6), | | | | |
| | | mRNA | | | | |
| GA_1843 | NM_000982 | 60s ribosomal protein I21 | 1 | 7 | 48 | 42 |
| GA_35369 | NM_003374 | Voltage-dependent anion-selective channel | 1 | 5 | 6 | 10 |
| | | (VDAC-1) | | | | |
| GA_23117 | NM_004772 | P311 protein [Homo sapiens] | 1 | 5 | 7 | 6 |
| GA_2597 | NM_138610 | Homo sapiens H2A histone family, member Y | 1 | 5 | 5 | 14 |
| | | (H2AFY), transcript variant 3, mRNA | | | | |
| GA_3283 | NM_004484 | Homo sapiens glypican 3 (GPC3), mRNA | 1 | 6 | 7 | 12 |
| GA_3530 | NM_002539 | Homo sapiens ornithine decarboxylase 1 | 1 | 10 | 8 | 9 |
| | | (ODC1), mRNA | | | | |
| GA_4145 | NM_002480 | Protein phosphatase 1, regulatory(inhibitor) | 1 | 6 | 6 | 6 |
| | | subunit 12A (PPP1R12A) | | | | |
| GA_5992 | NM_014899 | Homo sapiens Rho-related BTB domain | 0 | 10 | 7 | 13 |
| | | containing 3 (RHOBTB3), mRNA | | | | |
| GA_6136 | NM_016368 | Homo sapiens myo-inositol 1-phosphate | 1 | 7 | 5 | 16 |
| | | synthase A1 (ISYNA1), mRNA | | | | |
| GA_6165 | NM_015853 | Orf (LOC51035) | 1 | 5 | 9 | 5 |
| GA_6219 | NM_016139 | 16.7 Kd protein (LOC51142), | 1 | 5 | 13 | 14 |
| GA_723 | NM_005801 | Homo sapiens putative translation initiation | 1 | 14 | 15 | 19 |
| | | factor (SUI1), mRNA | | | | |
| GA_9196 | NM_000404 | Homo sapiens galactosidase, beta 1 (GLB1), | 0 | 6 | 10 | 7 |
| | | transcript variant 179423, mRNA | | | | |
| GA_9649 | NM_014604 | Tax interaction protein 1 (TIP-1) | 0 | 8 | 5 | 5 |

### Example 3

### Specificity of Expression Confirmed by Real-Time PCR

To verify the expression patterns of particular genes of interest at the mRNA level, extracts of undifferentiated hES cells and their differentiated progeny were assayed by real-time PCR. Cells were cultured for 1 week with 0.5% dimethyl sulfoxide (DMSO) or 500 nM retinoic acid (RA). The samples were amplified using sequence-specific primers, and the rate of amplification was correlated with the expression level of each gene in the cell population.

Taqman™ RT-PCR was performed under the following conditions: 1×RT Master Mix (ABI), 300 nM for each primer, and 80 nM of probe, and 10 pg to 100 ng of total RNA in nuclease-free water. The reaction was conducted under default RT-PCR conditions of 48° C. hold for 30 min, 95° C. hold for 10 min, and 40 cycles of 95° C at 15 sec and 60° C. hold for 1 min. RNA was isolated by a guanidinium isothiocyanate method (RNAeasy™ kit, Qiagen) according to manufacturer's instructions, and subsequently DNAse treated (DNAfree™ kit, Ambion). Gene-specific primers and probes were designed by PrimerExpress™ software (Ver. 1.5, ABI). Probe oligonucleotides were synthesized with the fluorescent indicators 6-carboxyfluorescein (FAM) and 6-carboxy-tetramethylrhodamine (TAMRA) at the 5′ and 3′ ends, respectively. Relative quantitation of gene expression between multiple samples was achieved by normalization against endogenous18S ribosomal RNA (primer and probe from ABI) using the ΔΔC_{T} method of quantitation (ABI). Fold change in expression level was calculated as 2^{ΔΔCT}.

The table below shows the results of this analysis. Since the cells have been cultured in RA and DMSO for a short period, they are at the early stages of differentiation, and the difference in expression level is less dramatic than it would be after further differentiation. Of particular interest for following or modulating the differentiation process are markers that show modified expression within the first week of differentiation by more than 2-fold (*), 5-fold (**), 10-fold (***), or 100-fold (****).

TABLE 4
Quantitative RT-PCR analysis of gene expression in hESC differentiation

| | | | | Fold Change | |
|---|---|---|---|---|---|
| | Geron ID | GenBank ID | Name | RA | DMSO |
| A. | GA_10902 | NM_024504 | Pr domain containing 14 (PRDM14) ** | −1.9 | −8.3 |
| | GA_11893 | NM_032805 | Hypothetical protein FLJ14549 *** | −2.3 | −10.0 |
| | GA_12318 | NM_032447 | Fibrillin3 | | |
| | GA_1322 | NM_000142 | Fibroblast growth factor receptor 3 precursor | 1.5 | 2.3 |
| | | | (FGFR-3) * | | |
| | GA_1329 | NM_002015 | Forkhead box o1a (foxo1a) * | −1.6 | −2.9 |
| | GA_1470 | NM_003740 | Potassium channel subfamily k member 5 (TASK-2) | −1.6 | 1.0 |
| | GA_1674 | NM_002701 | Octamer-binding transcription factor 3a (OCT-3A) | −3.7 | −7.7 |
| | | | (OCT-4) ** | | |
| | GA_2024 | NM_003212 | Teratocarcinoma-derived growth factor 1 | −4.0 | −12.5 |
| | | | (CRIPTO) *** | | |
| | GA_2149 | NM_003413 | Zic family member 3 (ZIC3) ** | −1.7 | −5.3 |
| | GA_2334 | NM_000216 | Kallmann syndrome 1 sequence (KAL1) * | −1.1 | −2.5 |
| | GA_23552 | BC027972 | Glypican-2 (cerebroglycan) | −1.5 | −1.2 |
| | GA_2356 | NM_002851 | Protein tyrosine phosphatase, receptor-type, z | −1.7 | −3.3 |
| | | | polypeptide 1 (PTPRZ1) * | | |
| | GA_2367 | NM_003923 | Forkhead box h1 (FOXH1) ** | −1.8 | −5.6 |
| | GA_2436 | NM_004329 | Bone morphogenetic protein receptor, type Ia | −2.4 | −2.4 |
| | | | (BMPR1A) (ALK-3) * | | |
| | GA_2442 | NM_004335 | Bone marrow stromal antigen 2 (BST-2) | 1.1 | −1.9 |
| | GA_2945 | NM_005232 | Ephrin type-a receptor 1 (EPHA1) | −1.3 | −1.9 |
| | GA_2962 | NM_005314 | Gastrin-releasing peptide receptor (GRP-R) ** | −6.3 | −9.1 |
| | GA_2988 | NM_005397 | Podocalyxin-like (PODXL) * | −2.6 | −4.3 |
| | GA_3337 | NM_006159 | Nell2 (NEL-like protein 2) | −1.3 | −1.3 |
| | GA_3559 | NM_005629 | Solute carrier family 6, member 8 (SLC6A8) | −1.1 | −1.1 |
| | GA_420 | X98834 | Zinc finger protein, HSAL2 * | −1.4 | −2.8 |
| | GA_5391 | NM_002968 | Sal-like 1 (SALL1), | 1.4 | −1.3 |
| | GA_6402 | NM_016089 | Krab-zinc finger protein SZF1-1 * | −1.8 | −3.1 |
| | GA_9167 | AF308602 | Notch 1 (N1) | 1.3 | 1.0 |
| | GA_9183 | AF193855 | Zinc finger protein of cerebellum ZIC2 * | 1.0 | −2.9 |
| | GA_9443 | NM_004426 | Early development regulator 1 (polyhomeotic 1 | −1.8 | −5.6 |
| | | | homolog) (EDR1) ** | | |
| B. | GA_9384 | NM_020997 | Left-right determination, factor b (LEFTB) ** | −16.7 | −25.0 |
| | GA_12173 | BC010641 | Gamma-aminobutyric acid (GABA) A receptor, | −2.8 | −5.6 |
| | | | beta 3 ** | | |
| | GA_10513 | NM_033209 | Thy-1 co-transcribed *** | −12.5 | −11.1 |
| | GA_1831 | NM_002941 | Roundabout, axon guidance receptor, homolog 1 | 1.1 | 1.0 |
| | GA_2753 | NM_000582 | (ROBO1), | −3.8 | −10.0 |
| | | | Secreted phosphoprotein 1 (osteopontin) *** | | |
| | GA_32919 | NM_133259 | 130 kDa leucine-rich protein (LRP 130) | −1.9 | −1.9 |
| | GA_28290 | AK055829 | FLJ31267 (acetylglucosaminyltransferase-like | −2.3 | −4.5 |
| | | | protein) * | | |
| C. | GA_28053 | T24677 | EST **** | <−100* | <−100* |
| | GA_26303 | NM_138815 | Hypothetical protein BC018070 *** | −3.2 | −10.0 |
| | GA_2028 | NM_003219 | Telomerase reverse transcriptase (TERT) * | −2.1 | −2.3 |

### Example 4

### Selection of Markers for Monitoring ES Cell Differentiation

Genes that undergo up- or down-regulation in expression levels during differentiation are of interest for a variety of different commercial applications, as described earlier. This experiment provides an example in which certain genes were selected as a means to monitor the ability of culture conditions to maintain the undifferentiated cell phenotype—and hence, the pluripotent differentiation capability of the cells.

Particular genes were chosen from those identified as having differential expression patterns, because they are known or suspected of producing a protein gene product that is expressed at the cell surface, or is secreted. These attributes are helpful, because they allow the condition of the cells to be monitored easily either by antibody staining of the cell surface, or by immunoassay of the culture supernatant. Genes were chosen from the EST database (Groups 1), microarray analysis (Group 2), and other sources (Group 3).

TABLE 5
Additional Genes analyzed by real-time PCR

| | | GenBank or |
|---|---|---|
| | Name | ID No. |
| Group 1 | Bone marrow stromal antigen | NM_004335 |
| | Podocalyxin-like | NM_005397 |
| | Rat GPC/glypican-2 (cerebroglycan) | TA_5416486 |
| | Potassium channel subfamily k member 5 | NM_003740 |
| | (TASK-2) | |
| | Notch 1 protein | AF308602 |
| | Teratocarcinoma-derived growth factor 1 | NM_003212 |
| | (Cripto) | |
| | Nel 1 like/NELL2 (Nel-like protein 2) | NM_006159 |
| | Gastrin releasing peptide receptor | NM_005314 |
| | Bone morphogenetic protein receptor | NM_004329 |
| | ABCG2- ABC transporter | AY017168 |
| | Solute carrier family 6, member 8 | NM_005629 |
| | (SLC6A8) | |
| | hTERT | NM_003219 |
| | Oct 3/4 octamer-binding transcription | NM_002701 |
| | factor 3a (oct-3a) (oct-4) | |
| Group 2 | Left-right determination factor b | NM_020997 |
| | (LEFTB) | |
| | Secreted phosphoprotein 1 (osteopontin) | NM_000582 |
| | Gamma-aminobutyric acid (GABA) A | NM_021912 |
| | receptor, beta 3 | |
| | Roundabout, axon guidance receptor, | NM_002941 |
| | homologue 1 (ROBO1), | |
| | Glucagon receptor | NM_00160 |
| | Leucine-rich PPR-motif hum 130 kDa | M92439 |
| | hum130leu 130kd Leu | |
| | Thy-1 co-transcribed | NM_033209 |
| | Solute carrier family 21 | NM_016354 |
| | LY6H lymphocyte antigen 6 complex | NM_002347 |
| | locus H | |
| | Plexin (PLXNB3) | NM_005393 |
| | ICAM | NM_000201 |
| Group 3 | Rhodopsin | NM_000539 |
| | Kallmann syndrome 1 sequence (KAL1) | NM_000216 |
| | Armadillo repeat protein deleted in | NM_001670 |
| | velo-cardio-facial syndrome (ARVCF) | |
| | Ephrin type-a receptor 1 (EPHA1) | NM_005232 |

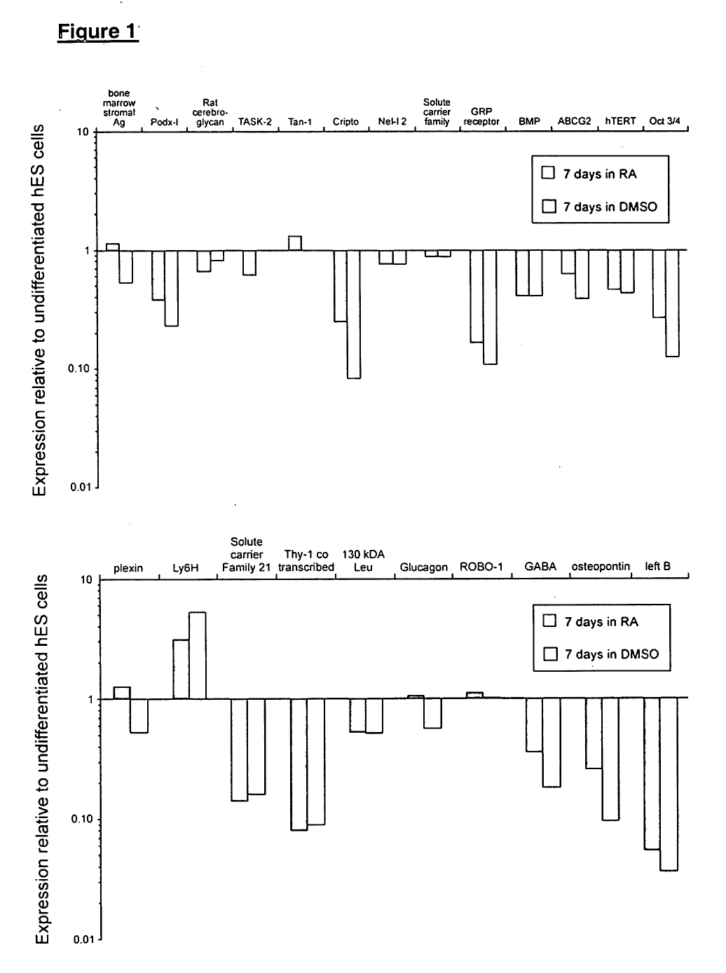

FIG. 1 shows the decrease in expression of the genes in Group I (Upper Panel) and Group II (Lower Panel) in H9 hES cells after culturing for 7 days with RA or DM. Gene expression of rhodopsin and ICAM was below the limit of detection in differentiated cells. KAL1 and EPHA1 were not tested.

Besides hTERT and Oct 3/4, three other genes were selected as characteristic of the undifferentiated hES cell phenotype. They were Teratocarcinoma-derived growth factor (Cripto), Podocalyxin-like (PODXL), and gastrin-releasing peptide receptor (GRPR).

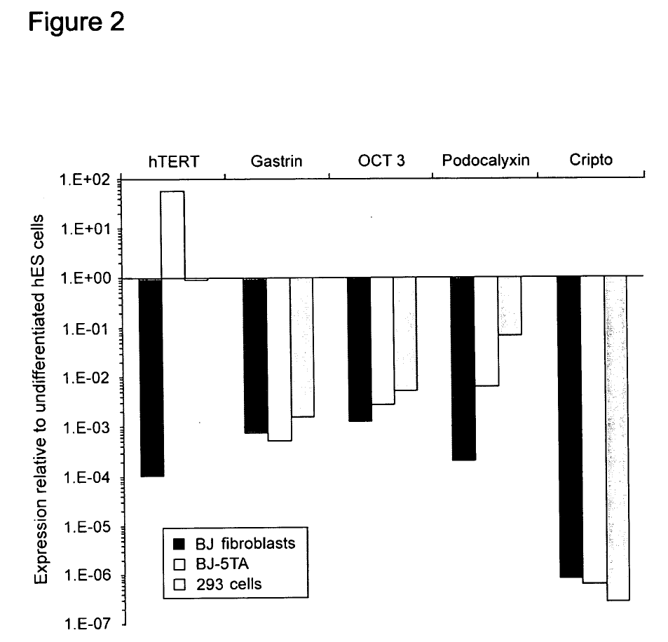

FIG. 2 compares the level of expression of these five genes in hES cells with fully differentiated cells: BJ fibroblasts, BJ fibroblasts transfected to express hTERT (BJ-5TA), and 293 (human embryonic kidney) cells. The level of all markers shown was at least 10-fold higher, and potentially more than 10², 10³, 10⁴, 10⁵, or 10⁶-fold higher in pluripotent stem cells than fully differentiated cells. All five markers retained a detectable level of expression in differentiated cultures of hESC. It is not clear if there is lower level of expression of these markers in differentiated cells, or if the detectable expression derived from the undifferentiated cells in the population. The one exception observed in this experiment was the hTERT transgene, expressed at an elevated level as expected in the BJ-5TA cells.

High-level expression of Cripto, GRPR and PODXL in undifferentiated hES cells reveals interesting aspects of the biology of these cells. Cripto has been implicated in normal mammalian development and tumor growth. Cripto encodes a glycosylphosphoinositol anchored protein that contains an EGF repeat and a cysteine rich motif, which makes it a member of the EGF-CFC family. It has been demonstrated that Cripto serves as a co receptor for Nodal, which is essential for mesoderm and endoderm formation in vertebrate development (Yeo et al., Molecular Cell 7:949, 2001). The finding that Cripto is expressed preferentially on undifferentiated hESC suggests that Nodal is an important signaling molecule for stem cells, perhaps to promote survival and/or proliferation.

PODXL encodes for transmembrane sialoprotein that is physically linked to the cytoskeleton. PODXL is suspected to act as an inhibitor of cell-cell adhesion and has been implicated in the embryonic development of the kidney podocyte. The anti-adhesion properties of PODXL when expressed on undifferentiated hESC may be an important feature related to stem cell migration.

The receptor for gastrin releasing peptide (GRP) is a G-protein coupled receptor that mediates numerous biological effects of Bombesin-like peptides, including regulation of gut acid secretion and satiety. A critical role has also been established for GRP and GRPR in control growth of cultured cells and normal mammalian development. GRP and GRPR may be oncofetal antigens that act as morphogens in normal development and cancer.

### Example 5

### Use of Cell Markers to Modify ES Cell Culture Conditions

This example illustrates the utility of the differentially expressed genes identified according to this invention in the evaluation of culture environments suitable for maintaining pluripotent stem cells.

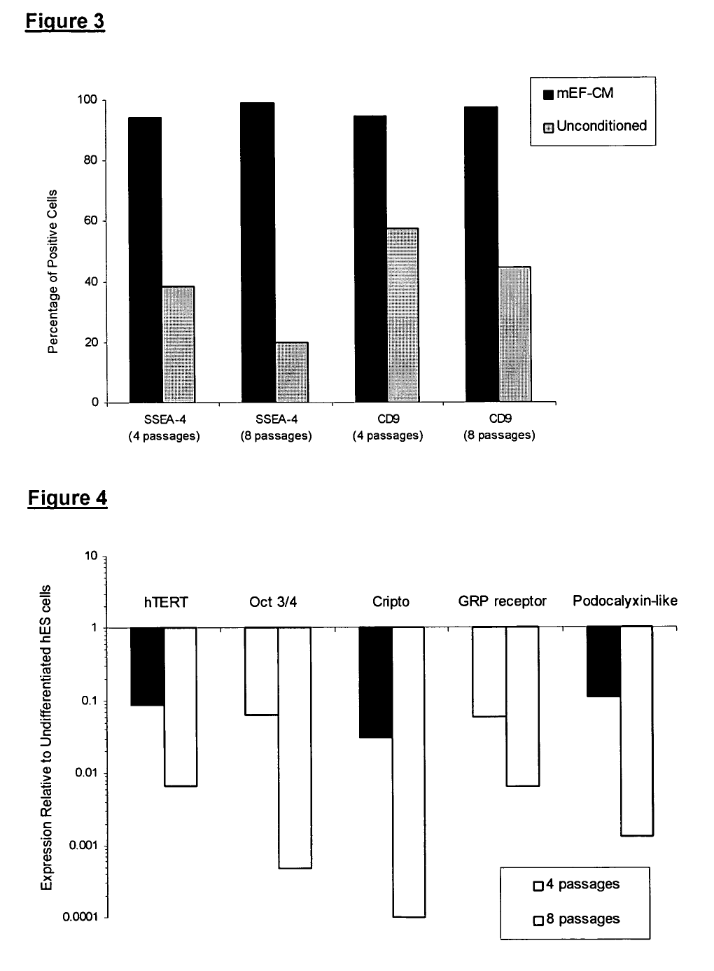

FIG. 3 show results of an experiment in which hES cells of the H1 line were maintained for multiple passages in different media. Medium conditioned with feeder cells provides factors effective to allow hES cells to proliferate in culture without differentiating. However, culturing in unconditioned medium leads to loss of the undifferentiated phenotype, with an increasing percentage of the cells showing decreased expression of CD9 (a marker for endothelial cells, fibroblasts, and certain progenitor cells), and the classic hES cell marker SSEA-4.

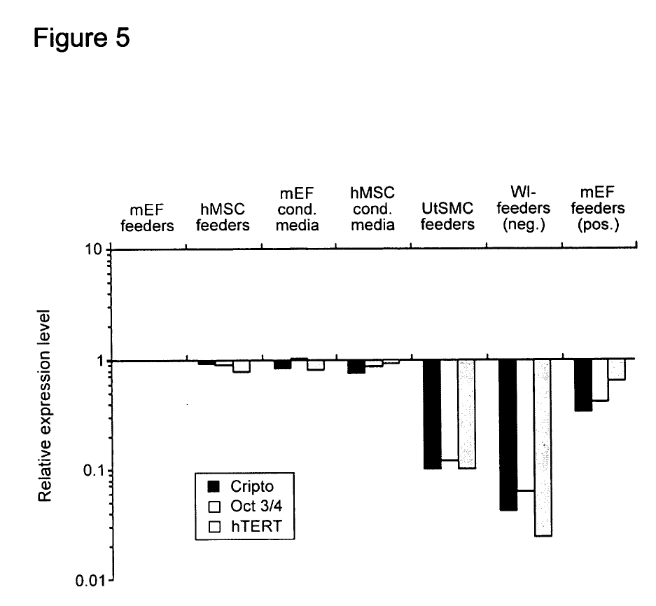

FIG. 4 illustrates the sensitivity of hTERT, Oct 3/4, Cripto, GRP receptor, and podocalyxin-like protein (measured by real-time PCR assay) as a means of determining the degree of differentiation of the cells. After 4 passages in unconditioned X-VIVO™ 10 medium containing 8 ng/mL bFGF, all 5 markers show expression that has been downregulated by about 10-fold. After 8 passages, expression has decreased by 10², 10³, or 10⁴-fold.

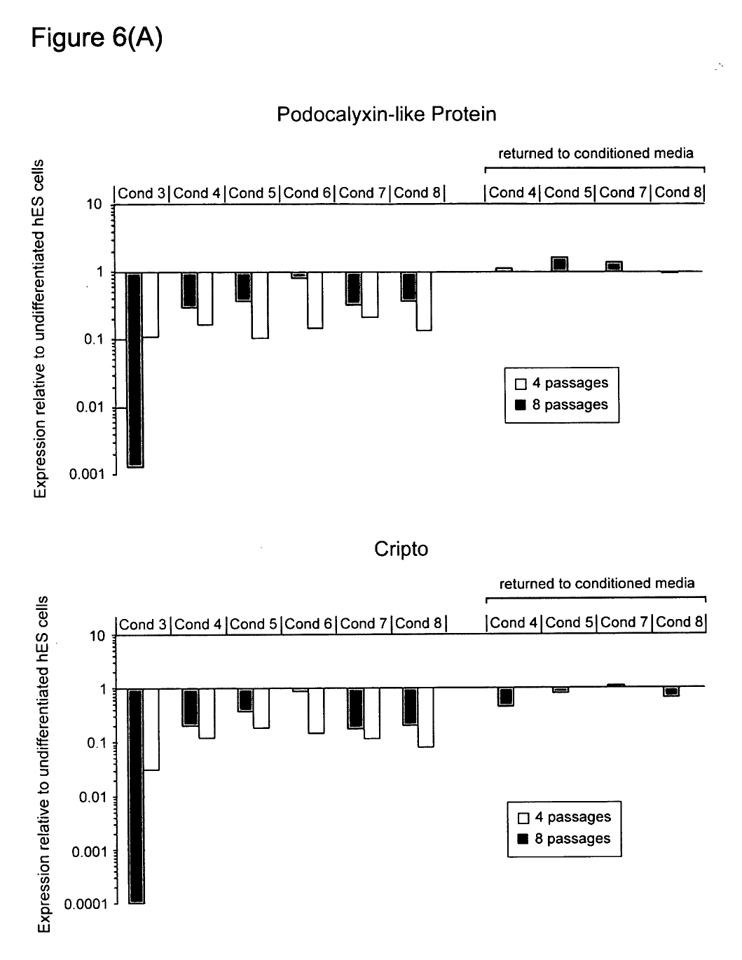

FIG. 5 shows results of an experiment in which the hES cell line H1 was grown on different feeder cell lines: mEF=mouse embryonic fibroblasts; hMSC=human mesenchymal stem cells; UtSMC=human uterine smooth muscle cells; WI-38=an established line of human lung fibroblasts. As monitored by RT-PCR assay of Cripto, Oct 3/4, and hTERT, at least under the conditions used in this experiment, the hMSC are better substitutes for mEF feeders than the other cell lines tested.

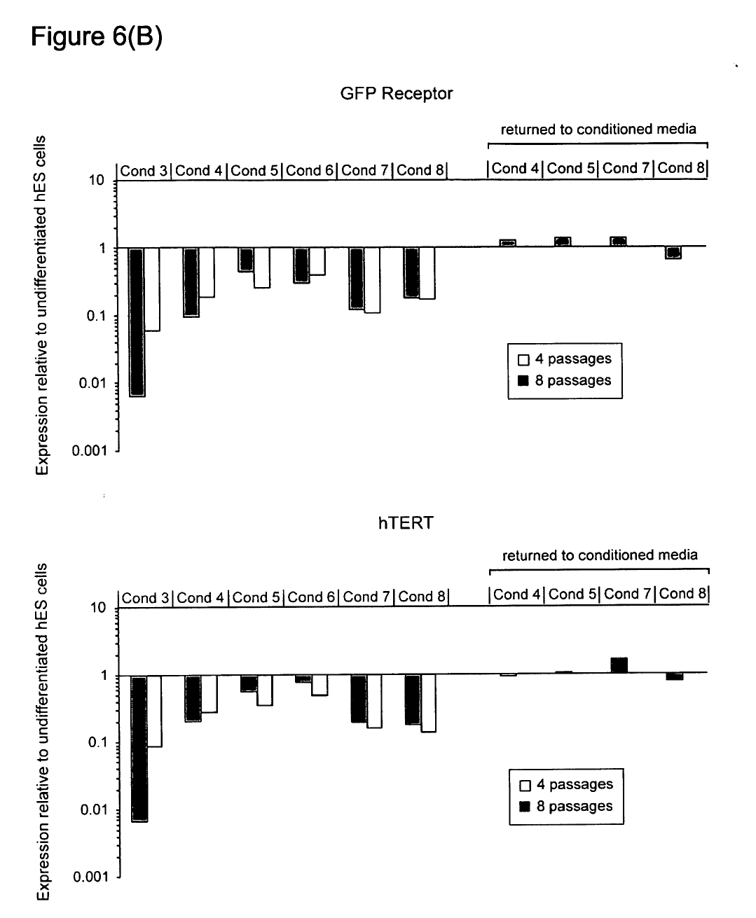

FIG. 6 shows results of an experiment in which different media were tested for their ability to promote growth of hES cells without differentiation. Expression of Podocalyxin-like protein, Cripto, GFP Receptor, and hTERT were measured by RT-PCR. The test media were not preconditioned, but supplemented with the growth factors as follows:

TABLE 6
Growth Conditions Tested for Marker Expression

| | DMEM preconditioned with mEF + |
|---|---|
| Standard conditions: | bFGF (8 ng/mL) |
| Condition 3 | X-VIVO ™ 10 + bFGF (8 ng/mL) |
| Condition 4 | X-VIVO ™ 10 + bFGF (40 ng/mL) |
| Condition 5 | X-VIVO ™ 10 + bFGF (40 ng/mL) + |
| | stem cell factor (SCF, 15 ng/mL) |
| Condition 6 | X-VIVO ™ 10 + bFGF (40 ng/mL) + |
| | Flt3 ligand (75 ng/mL) |
| Condition 7 | X-VIVO ™ 10 + bFGF (40 ng/mL) + |
| | LIF (100 ng/mL) |
| Condition 8 | QBSF ™-60 + bFGF (40 ng/mL) |

The results show that the markers selected to monitor the undifferentiated phenotype showed similar changes in each of these culture conditions. By all criteria, XVIVO 10™ supplemented according to Condition 6 was found to be suitable for culturing hES cells without having to be preconditioned. As shown on the right side, when cells were put back into standard conditioned medium after 8 passages in the test conditions, expression of all four markers returned essentially to original levels. This shows that alterations in expression profiles in media Conditions 4 to 8 are temporary and reversible—consistent with the cells retaining full pluripotency.

### Example 6

### Measuring Undifferentiated Cell Markers by Flow Cytometry

Cells from the undifferentiated hES cell line H1 were grown in mEF conditioned medium in Matrigel® coated 6-well plates. Cells were harvested using 3.0 mL of 0.5 mM EDTA and resuspended in PBS containing 5% fetal calf serum and 0.05% NaN₃ at a concentration of 5×10⁶ cells/mL. For SSEA-4 and TRA1-60 staining, 1 μg of antibody (Chemicon International) was used. Cells were incubated for a period of 30 min on ice followed by one wash with 2.0 mL of PBS-FCS buffer. Cell pellets were resuspended in 100 μl of fluorochrome conjugated secondary antibody. For intracellular Oct-4 staining, the cells were fixed with 2% PFA (final concentration) for 15 min at room temperature. After one wash, cells were resuspended in a permeabilization buffer (PBS-FCS plus 90% cold methanol) followed by 15 min in ice, washed again, and then resuspended the cell pellet in blocking solution (20% goat serum in permeabilization buffer). 0.5×10⁶ or 1.0×10⁶ permeabilized cells were stained with 1 μg of anti-Oct-4 antibody (Santa Cruz Biotechnology) in 10 μL of blocking solution, incubated on ice for 30 min. After rewashing, the cells were stained with labeled secondary antibody.

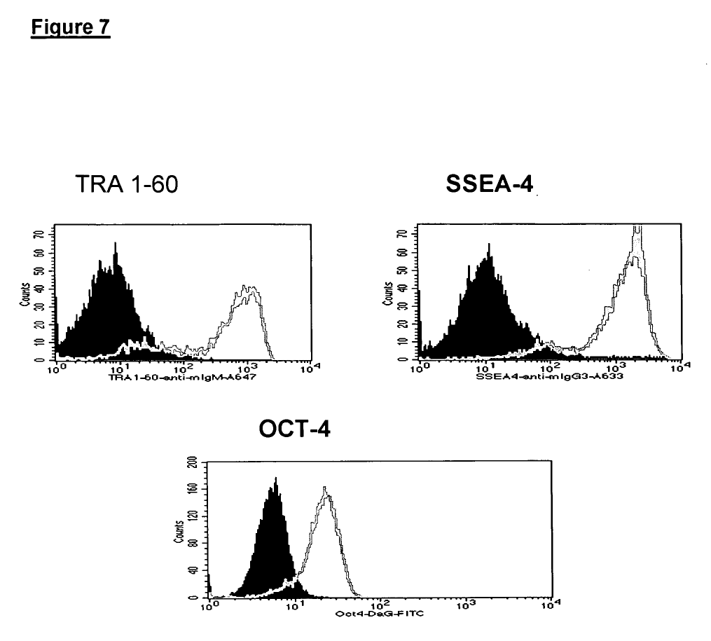

FIG. 7 shows that SSEA-4, TRA 1-60 and Oct-4 markers were all strongly expressed on undifferentiated cells under these conditions. Solid areas in each panel indicate background staining observed with the respective isotype-matched controls. In fact, greater than 85% of hES cells expressed all three markers.

### Example 7

### Measuring Differentiated Cells Using Stromal Markers

The extent of differentiation can be determined by detecting or measuring markers for undifferentiated cells, in combination with markers for differentiated cells of the type expected in early differentiation cultures—either by antibody staining, or by PCR amplification (Taqman™), or by a combination of techniques.

In this example, screening of useful stromal cell markers was done by immunocytochemistry of hES cells cultured in XVIVO 10™ with bFGF, or medium conditioned using mouse embryonic fibroblasts. Antibodies were obtained from commercial sources as follows:

TABLE 7
Primary Antibody for Measuring Differentiated Cells

| Marker | Vendor | Catalog No. |
|---|---|---|
| STRO-1 | RnD Systems | MAB 1038 |
| Human Thymus Stroma | BD Pharmingen | 555825 |
| CD44 | BD Pharmingen | 550988 |
| CD90 | BD Pharmingen | 555593 |
| CD105 (Endoglin) | Chemicon | MAB2152 |
| CD106 (VCAM-1) | BD Pharmingen | 555645 |
| Vimentin | Sigma | V 5255 |

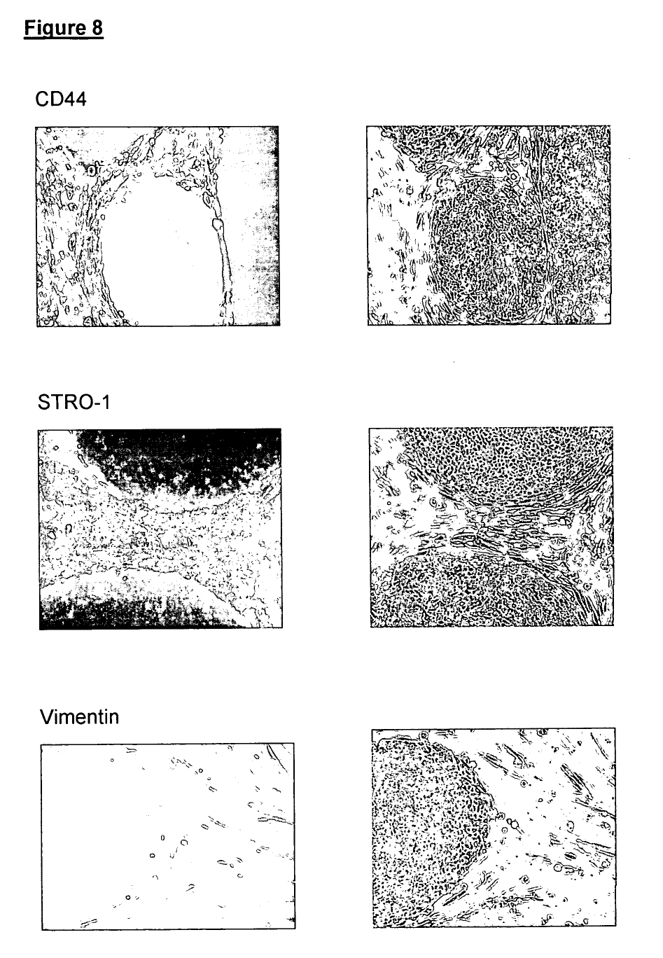

FIG. 8 shows the results of the immunocytochemical analysis. CD44, STRO-1 and Vimentin stain stromal-like cells in the hES cell populations cultured with mEF conditioned medium.

### Example 8

### Sensitivity of the Assay for Undifferentiated Cells

Real-time PCR assays were performed using mixtures of undifferentiated hES cells and BJ fibroblasts, to determine the sensitivity of the assay for the presence of differentiated cells.

Freshly harvested cells were combined to a total of 2×10⁶ cells in 10% increments of each cell type. Total RNA was isolated (Roche isolation kit), and then treated with DNAse 1 to remove potential DNA contaminants. (Ambion kit). Amplification mixtures were made up in QRT-PCR master mix buffer (P/N 4309169) to a final volume of 25 μL at a concentration of 10 μM forward primer, 10 μM reverse primer, 10 μM probe, and ≠100 ng RNA. Data analysis was performed using the comparative Ct method using 18S rRNA endogenous control. (Other suitable housekeeping genes for standardization can be used instead, such as acidic ribosomal protein, β-actin, cyclophilin, G3P dehydrogenase, or β2-microglobulin).

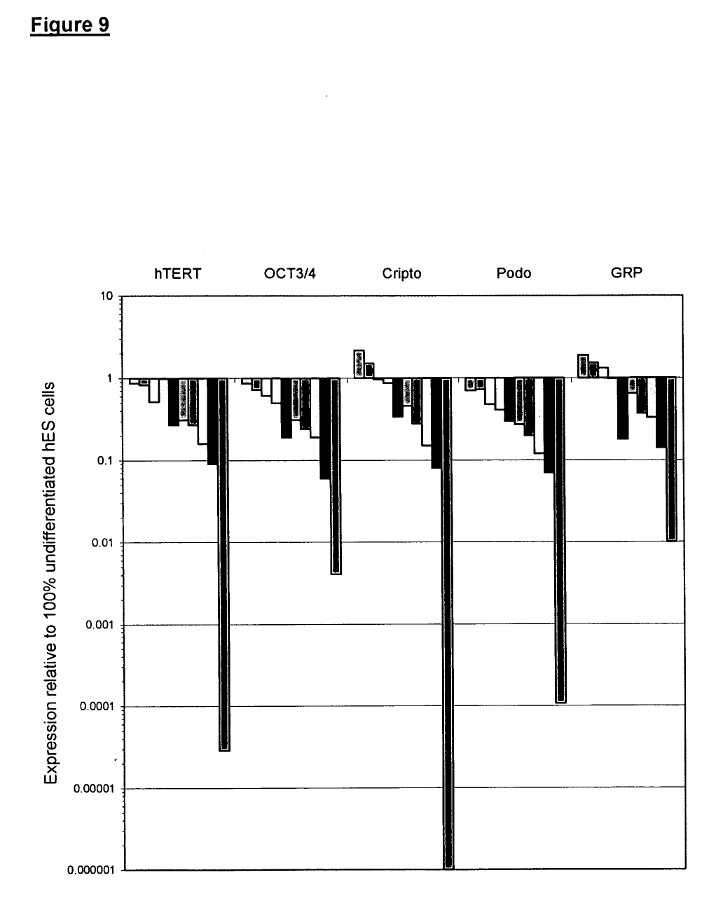

FIG. 9 shows the relative change of gene expression measured in mixtures of differentiated (BJ) and undifferentiated hES cells, compared with undifferentiated hES cells alone. These five markers are able to rank 10% changes in the proportion of undifferentiated cells.

TABLE 8
Sequences Referred To in this Disclosure

| Designation | Reference |
|---|---|
| hTERT mRNA sequence | GenBank Accession NM_003129 |
| hTERT protein sequence | GenBank Accession NM_003129 |
| Oct 3/4 mRNA sequence | GenBank Accession NM_002701 |
| Oct 3/4 protein sequence | GenBank Accession NM_002701 |
| Cripto mRNA sequence | GenBank Accession NM_003212 |
| Cripto protein sequence | GenBank Accession NM_003212 |
| podocalyxin-like protein | GenBank Accession NM_005397 |
| mRNA sequence | |
| podocalyxin-like protein | GenBank Accession NM_005397 |
| amino acid sequence | |
| GRP receptor mRNA sequence | GenBank Accession NM_005314 |
| GRP receptor proteins | GenBank Accession NM_005314 |
| sequence | |
| CD44 antigen mRNA sequence | GenBank Accession NM_000610 |
| (homing function and | |
| Indian blood group system) | |
| CD44 protein sequence | GenBank Accession NM_000610 |
| Vimentin mRNA sequence | GenBank Accession BC066956 |
| Vimentin protein sequence | GenBank Accession BC066956 |

The subject matter provided in this disclosure can be modified as a matter of routine optimization, without departing from the spirit of the invention, or the scope of the appended claims.

1. A method for determining the extent of differentiation in a population of isolated human embryonic stem (hES) cells, comprising detecting or measuring two or more markers preferentially expressed in undifferentiated hES cells, and one or more markers preferentially expressed after differentiation of the hES cells.

2. The method of claim 1, wherein at least one of the markers preferentially expressed in undifferentiated hES cells is selected from Cripto, gastrin-releasing peptide (GRP) receptor, podocalyxin-like protein (PODXL), and human telomerase reverse transcriptase (hTERT).

3. The method of claim 1, wherein at least one of the markers preferentially expressed in undifferentiated hES cells is selected from Oct 3/4, SSEA-4, and the markers detected by antibodies Tra-1-60 and Tra-1-81.

4. The method of claim 1, comprising measuring three or more markers preferentially expressed in undifferentiated hES cells selected from hTERT, Oct 3/4, Cripto, GRP, PODXL, SSEA-3, SSEA-4, Tra-1-60 and Tra-1-81.

5. The method of claim 1, comprising detecting or measuring hTERT, Oct 3/4, and a marker selected from Cripto, SSEA-4, Tra-1-60 and Tra-1-81.

6. The method of claim 1, wherein at least one of the markers preferentially expressed after differentiation of the hES cells is a stromal cell markers.

7. The method of claim 1, wherein the stromal cell marker is selected from CD44, CD105 (endoglin), CD106 (VCAM-1), CD90 (Thy-1), STRO-1, Vimentin, and Human Thymus Stroma.

8. The method of claim 1, wherein expression of hTERT, Oct 3/4, Cripto, GRP receptor, PODXL, CD44, CD105, CD106, or CD90 is detected or measured at the mRNA level by PCR amplification.

9. The method of claim 8, wherein the measuring at the mRNA level is conducted by real-time PCR amplification.

10. The method of claim 1, wherein expression of SSEA-3, SSEA-4, Tra-1-60, Tra-1-81, Cripto, Oct 3/4, CD44, CD105, CD106, CD90, STRO-1, Vimentin, or Human Thymus Stroma is detected or measured at the antigen expression level by antibody assay.

11. The method of claim 10, wherein the measuring at the antigen expression level is conducted by flow cytometry using fluorescence-labeled antibody.

12. The method of claim 10, wherein the measuring at the antigen expression level is conducted by immunocytochemistry.

13. The method of claim 1, comprising measuring some of said markers at the mRNA level, and some of said markers at the antigen expression level.

14. The method of claim 1, comprising quantifying the proportion of undifferentiated hES cells or differentiated cells in the culture from said marker expression according to positive expression of the undifferentiated cell markers, and lack of expression of the stromal cell markers.

15. The method of claim 1, comprising assessing the ability of a soluble factor or culture medium to maintain hES cells in an undifferentiated state from said marker expression.

16. The method of claim 1, comprising assessing the suitability of an undifferentiated hES cell population for preparing differentiated cells for human administration.

17. A system for assessing a culture of undifferentiated human embryonic stem (hES) cells or their progeny according to claim 1, comprising antibody or PCR amplification primers specific for three or more markers, of which at least two are preferentially expressed in undifferentiated hES cells, and at least one is preferentially expressed in stromal cells.

18. The system of claim 17, comprising antibody or PCR amplification primers specific for at least two markers selected from Cripto, gastrin-releasing peptide (GRP) receptor, podocalyxin-like protein (PODXL), human telomerase reverse transcriptase (hTERT) Oct 3/4, SSEA-4, and the markers detected by antibodies Tra-1-60 and Tra-1-81.

19. The system of claim 17, comprising antibody or PCR amplification primers specific for at least one stromal cell marker selected from CD44, CD105 (endoglin), CD106 (VCAM-1), CD90 (Thy-1), STRO-1, Vimentin, and Human Thymus Stroma.

20. The system of claim 17, packaged as a kit with instructions for using the components of the kit for assessing a culture of undifferentiated human embryonic stem (hES) cells.
